(19) United States
(12) Patent Application Publication
(10) Pub. No.: US 2026/0273301 A1
(43) Pub. Date: Sep. 17, 2026

(54) ENCAPSULATED NEGATIVE MAGNETIC DEVICE

(71) Applicant: Chih-Chiang HSU, New Taipei City (TW)

(72) Inventor: Chih-Chiang HSU, New Taipei City (TW)

(21) Appl. No.: 19/076,117

(22) Filed: Mar. 11, 2025

(51) Int. Cl. A61N 2/06

(52) U.S. Cl. CPC A61N 2/06

(57) ABSTRACT

An encapsulated negative magnetic device comprises an encapsulation body and a plurality of first magnetic elements. The encapsulation body is made of a flexible material, and it has an S-pole corresponding surface and an N-pole corresponding surface. The first magnetic elements are encapsulated in the encapsulation body, and each of the first magnetic elements has a first S-pole surface and a first N-pole surface. The first S-pole surfaces are located on the same side of the S-pole corresponding surface, and the first N-pole surfaces are located on the same side of the N-pole corresponding surface. Accordingly, the S-pole corresponding surface is attached to human skin, especially the affected part, so that the negative magnetic effect of the first magnetic elements inside it can provide the affected part with the effects of restoring, alkalizing, relaxing, calming, and resisting pressure.

### BACKGROUND OF THE INVENTION

### 1. Field of the Invention

The present invention is related to an encapsulated negative magnetic device, in particular to a negative magnetic device that can be attached to a part of the human body. The negative magnetic device brings positive benefits to the human body through a magnetic field.

### 2. Description of the Prior Art

Dr. Albert Roy Davis, an American biophysicist, discovered that the cosmic magnetic poles have two completely different effects on living things. The positive magnetic field effect is positively charged, and has the effects such as oxidation, acidification, activation, excitement, and increased pressure. The negative magnetic field effect is negatively charged, and has the effects such as reduction, alkalinity, relaxation, calmness, and stress resistance.

According to the Biot-Savart law, when the biomagnetic field of the human body adapts to the great magnetic field of the universe, it can maintain the normal physiology of the body's tissues and organs. Otherwise, abnormal reactions or illness will occur. Whenever a part of the human body encounters different types of traumas, the electric and magnetic fields of that part are always positive. Through the mechanism of the meridians and nervous system, the body will concentrate the negative magnetic energy of the body to reach the affected part, in order to achieve healing. Because there is no oxygen assistance in an acidic environment, the maintenance process must be in an alkaline environment so as to achieve good results.

To use magnets for health care, the magnets are placed as close to the human body as possible. However, the human body is not a flat surface. Different parts of the body have different curves, and each person's curves are also different. Therefore, designing a negative magnetic device that can bring the magnet close to each person's body curve is a topic worth studying.

### SUMMARY OF THE INVENTION

The present invention provides an encapsulated negative magnetic device, which combines a magnet with an encapsulation structure of a flexible material. Since the flexible material has good elasticity and softness, the encapsulation structure can easily fit tightly with the skin surface at each location, and is suitable for each part of the human body. Accordingly, the magnet can be placed close to each person's body curve.

The encapsulated negative magnetic device of the present invention comprises an encapsulation and a plurality of first magnetic component. The encapsulation body is made of flexible material, and has an S-pole corresponding surface and an N-pole corresponding surface. The first magnetic elements are encapsulated in the encapsulation body, and the first magnetic elements all have a first S-pole surface and a first N-pole surface. The first S-pole surfaces are located on the same side of the S-pole corresponding surface, and the first N-pole surfaces are located on the same side of the N-pole corresponding surface. Accordingly, the S-pole corresponding surface is attached to human skin, especially the affected part, so that the negative magnetic effect of the first magnetic elements inside it can provide the affected part with the effects of restoring, alkalizing, relaxing, calming, and resisting pressure.

In a preferred embodiment, the flexible material of the encapsulation body is silicone, and the first magnetic elements are permanent magnets.

In a preferred embodiment, a longitudinal spacing is provided between every two longitudinally adjacent magnetic elements, and a transverse spacing is provided between every two transversely adjacent first magnetic elements.

In a preferred embodiment, an S-pole effective magnetic spacing is provided between the first S-pole surfaces and the S-pole corresponding surface. The S-pole effective magnetic spacing is not greater than 10 mm (including 0.5 mm, 1 mm, 2 mm, 3 mm, 4 mm, 5 mm, 6 mm, 7 mm, 8 mm, 9 mm, 10 mm). A N-pole effective magnetic spacing is provided between the first N-pole surfaces and the N-pole corresponding surface. The N-pole effective magnetic spacing is not greater than 10 mm (including 0.5 mm, 1 mm, 2 mm, 3 mm, 4 mm, 5 mm, 6 mm, 7 mm, 8 mm, 9 mm, 10 mm).

In a preferred embodiment, the encapsulation body comprises at least one slot, the slots do not penetrate the encapsulation body, the slots are used to fix a vibration device, the vibration device comprises a vibration motor and a power supply, the vibration motor is electrically connected to the power supply, and the vibration motor can be inserted into a slot.

In a preferred embodiment, the encapsulation body comprises at least one inserting hole, the inserting holes all penetrate the encapsulation body, the inserting holes are used to fix a vibration device or a bandage, the vibration device comprises a vibration motor and a power source, the vibration motor is electrically connected to the power supply, and the vibration motor can be inserted into an inserting hole.

In a preferred embodiment, it further comprises an additional encapsulation body A material of the additional encapsulation body is a flexible material, the additional encapsulation body comprises a S-pole corresponding surface, an N-pole corresponding surface and at least one fixed slot, the additional encapsulation body encapsulates at least one second magnetic element, the second magnetic elements comprise a second S-pole surface and a second N-pole surface, the second S-pole surfaces are all located on the same side of the S-pole corresponding surface of the additional encapsulation body, the second N-pole surfaces are all located on the same side of the N-pole corresponding surface of the additional encapsulation body, the fixed slots are used to fix a vibration device, the vibration device comprises a vibration motor and a power supply, the vibration motor is electrically connected to the power supply, and the vibration motor can be inserted into a fixed slot.

In a preferred embodiment, an N-pole effective magnetic spacing is provided between the second N-pole surfaces and the N-pole corresponding surface of the additional encapsulation body, and the N-pole effective magnetic spacing is not greater than 10 mm (including 0.5 mm, 1 mm, 2 mm, 3 mm, 4 mm, 5 mm, 6 mm, 7 mm, 8 mm, 9 mm, 10 mm).

In a preferred embodiment, the flexible material of the additional encapsulation body is silicone, and the second magnetic elements are permanent magnets.

Based on the foregoing, the first magnetic element is encapsulated in the encapsulation body by the present invention. Since the encapsulation body is made of flexible material, when the encapsulation body is attached to human skin, it can easily fit tightly to the skin surface at each location. It is suitable for each part of the human body and is conducive to placing the first magnetic element close to the curves of each part of the human body.

### BRIEF DESCRIPTION OF THE DRAWINGS

FIG. 1 is a three-dimensional schematic diagram of the first embodiment of the present invention.

FIG. 2 is a cross-sectional schematic diagram of the first embodiment of the present invention.

FIG. 3 is a three-dimensional schematic diagram of the second embodiment of the present invention.

FIG. 4 is a cross-sectional schematic diagram of the second embodiment of the present invention.

FIG. 5 is a three-dimensional schematic diagram of the third embodiment of the present invention.

FIG. 6 is a cross-sectional schematic diagram of the third embodiment of the present invention.

FIG. 7 is a three-dimensional schematic diagram of another configuration of the third embodiment of the present invention.

FIG. 8A is a three-dimensional schematic diagram of the fourth embodiment of the present invention.

FIG. 8B is another three-dimensional schematic diagram of the fourth embodiment of the present invention.

FIG. 9A is a cross-sectional schematic diagram of the fourth embodiment of the present invention.

FIG. 9B is another cross-sectional schematic diagram of the fourth embodiment of the present invention.

FIG. 10A is three-dimensional schematic diagram of another configuration of the fourth embodiment of the present invention.

FIG. 10B is a three-dimensional schematic diagram of another configuration of the fourth embodiment of the present invention.

FIG. 11 is a three-dimensional schematic diagram of the head guard made of the encapsulation body of the present invention.

FIG. 12 is a three-dimensional schematic diagram of the wrist guard made from the encapsulation body of the present invention.

### DETAILED DESCRIPTION OF THE INVENTION

Other technical contents, features and effects of the present invention will be clearly presented in the following detailed description of the preferred embodiments with reference to the drawings. For the purpose of simplicity and forcefulness in the drawing, if it does not affect the interpretation of the technical characteristics of the patent, some of the structures and parts that are customarily used in the drawing will be shown in a simple and schematic manner, or omitted. In addition, the sizes of the structures and parts in the figure will be drawn in a scale suitable for readers to view, not the actual prototype scale.

Unless otherwise defined, all technical and scientific terms used herein have the same meaning as commonly understood by those skilled in the art to which this work belongs.

As used herein, the grammatical articles “a,” “an,” and “any” refer to one or more than one (i.e., at least one) of an item without specifying a particular number. For example, “an element” means one element or more than one element.

As used herein, the terms “first”, “second”, etc. are only used to distinguish between the described elements and should not be understood as indicating or implying relative importance, order of use, or order of arrangement.

As used herein, the terms “top”, “bottom”, “front”, “back”, “left”, “right”, “side”, “top”, and “bottom” describing the names of the components or their positional relationships are used to help describe the positional relationships between the featured structures, and the actual orientation of the featured structures may change depending on the angle of placement or the user's location, and do not limit the scope of this document to be equally covered.

The present invention provides an encapsulated negative magnetic device 10, which is a structure that can be attached to the human body. It has multiple implementation configurations. The following describes the detailed configuration of the structure of different forms through various embodiments.

### First Embodiment

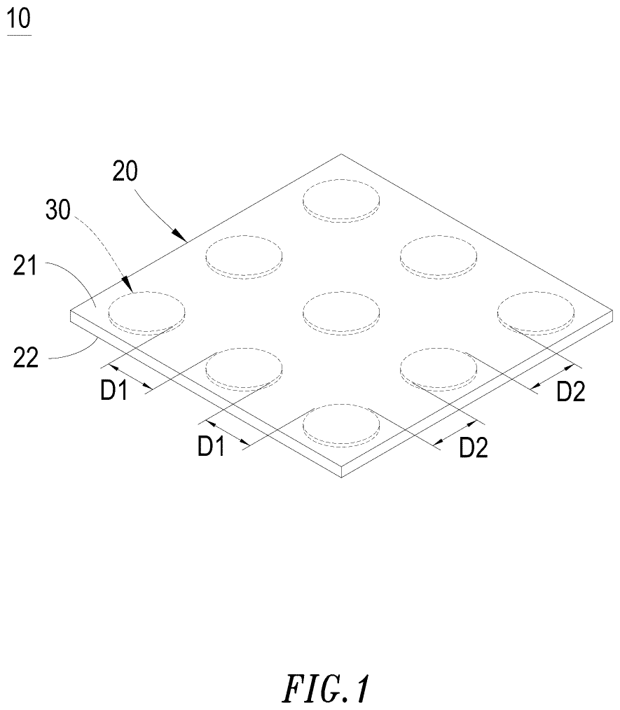

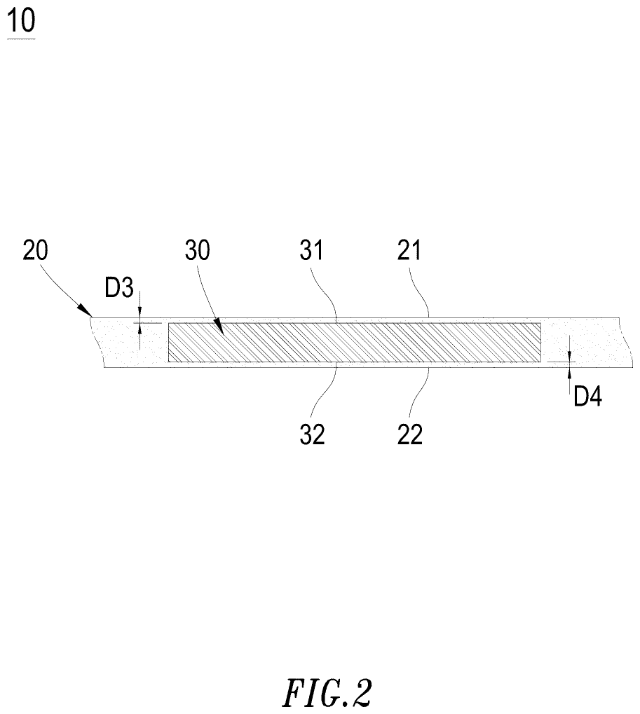

Please refer to FIGS. 1 and 2. FIG. 1 is a three-dimensional schematic diagram of the first embodiment of the present invention, and FIG. 2 is a cross-sectional schematic diagram of the first embodiment of the present invention. As shown in the figure, the encapsulated negative magnetic device 10 includes an encapsulation body 20 and a plurality of first magnetic elements 30.

The encapsulation body 20 is made of silicone and has an S-pole corresponding surface 21 and an N-pole corresponding surface 22. The S-pole corresponding surface 21 and the N-pole corresponding surface 22 are located on two opposite side surfaces of the encapsulation body 20. A plurality of first magnetic elements 30 are disposed inside the encapsulation body 20. In one embodiment, each first magnetic element 30 is evenly arranged in the encapsulation body 20. Accordingly, there is a longitudinal spacing D1 between every two longitudinally adjacent first magnetic elements 30, and there is a transverse spacing D2 between every two transversely adjacent first magnetic elements 30. Since a longitudinal spacing D1 or a transverse spacing D2 is provided between every two adjacent first magnetic elements 30, the first magnetic elements 30 can be effectively separated, thereby preventing the first magnetic elements 30 from attracting each other. Each first magnetic element 30 is a permanent magnet, and each first magnetic element 30 has a first S-pole surface 31 and a first N-pole surface 32.

In one embodiment, each first S-pole surface 31 is located on the same side of the S-pole corresponding surface 21. In order to prevent each first magnetic element 30 from losing its magnetic attraction effect due to the isolation of the encapsulation body 20, a S-pole effective magnetic spacing D3 is provided between each first S-pole surface 31 and the S-pole corresponding surface 21, and the S-pole effective magnetic spacing D3 is not greater than 10 mm. As such, the encapsulation body 20 can enclose each first magnetic element 30 and also maintain the magnetic attraction effect of each first magnetic element 30. That is, each first N-pole surface 32 is located on the same side of the N-pole corresponding surface 22. In order to prevent each first magnetic element 30 from losing its magnetic attraction effect due to the isolation of the encapsulation body 20, a N-pole effective magnetic spacing D4 is provided between each first N-pole surface 32 and the N-pole corresponding surface 22, and the N-pole effective magnetic spacing D4 is not greater than 10 mm. As such, the encapsulation body 20 can enclose each first magnetic element 30 and also maintain the magnetic attraction effect of each first magnetic element 30.

When in use, the S-pole corresponding surface 21 is attached to human skin, especially the affected part. Accordingly, the negative magnetic effect of the first magnetic element 30 inside it can provide the affected part with the effects of restoring, alkalizing, relaxing, calming, and resisting pressure. In other embodiments, the N-pole corresponding surface 22 may also be attached to human skin, but this embodiment is not limited thereto.

### Second Embodiment

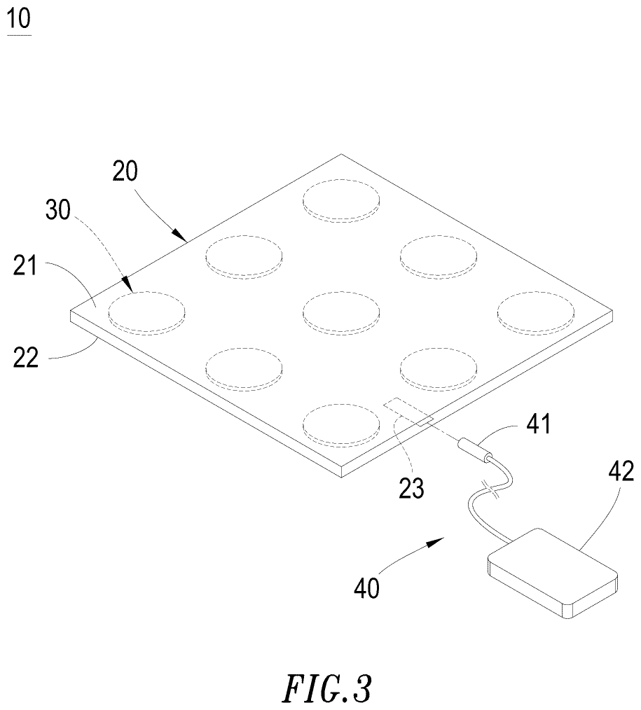

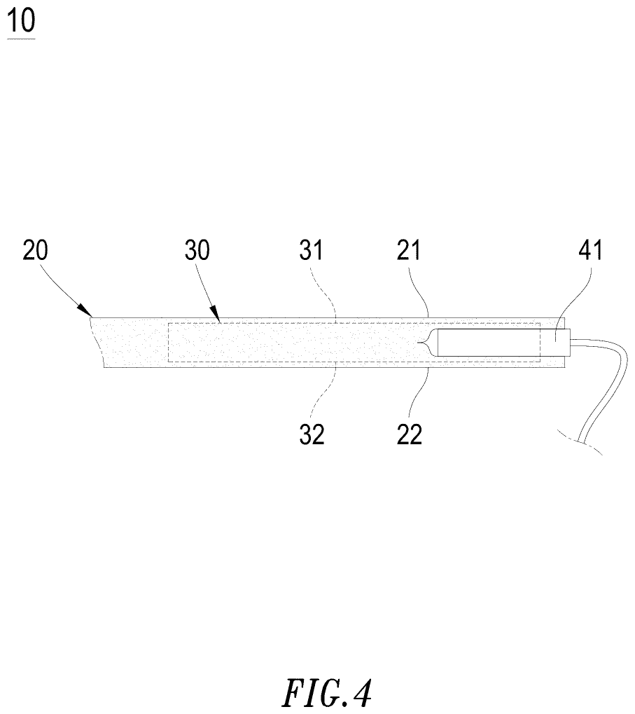

Please refer to FIGS. 3 and 4. FIG. 3 is a three-dimensional schematic diagram of the second embodiment of the present invention, and FIG. 4 is a cross-sectional schematic diagram of the second embodiment of the present invention. As shown in the figure, the encapsulated negative magnetic device 10 includes an encapsulation body 20, a plurality of first magnetic elements 30 and a vibration device 40.

The present embodiment differs from the first embodiment in that, the present embodiment further includes a vibration device 40, and the encapsulating body 20 includes a slot 23. The remaining structures are the same as those of the first embodiment, and thus will not be described in detail. The slot 23 does not penetrate the encapsulating body 20. In other words, the slot 23 has only one opening on the encapsulating body 20. Regarding the vibration device 40, it includes a vibration motor 41 and a power supply 42. The vibration motor 41 is, for example, but not limited to, any one of an electric motor, a pneumatic motor or a hydraulic motor. The vibration motor 41 is electrically connected to the power supply 42. The power supply 42 is any one of an external power supply and a built-in power supply. The built-in power supply is electrically connected to a circuit board. The circuit board is used to control the power supply mechanism of the built-in power supply, so as to adjust the vibration time or vibration intensity of the vibration motor 41 by adjusting the power supply time or the power supply current. Therefore, when in use, the vibration motor 41 can be inserted into the slot 23, and the S-pole corresponding surface 21 is attached to the human skin, especially the affected part. The negative magnetic effect provides the affected part with the effects of restoring, alkalizing, relaxing, calming, and resisting pressure. When the vibration motor 41 is operated, each first magnetic element 30 can be vibrated to expand the magnetic field function. By striking each first magnetic element 30, the magnetic field can be strengthened to penetrate into the skin and muscles.

### Third Embodiment

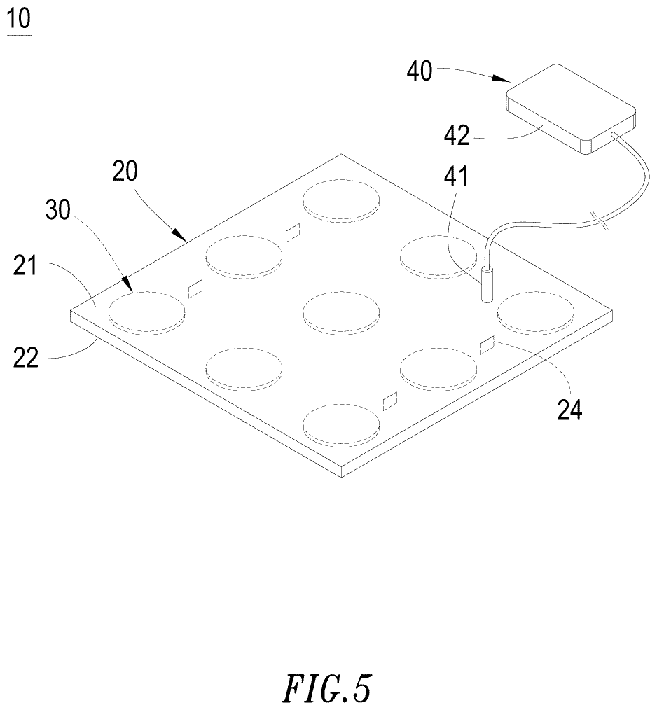

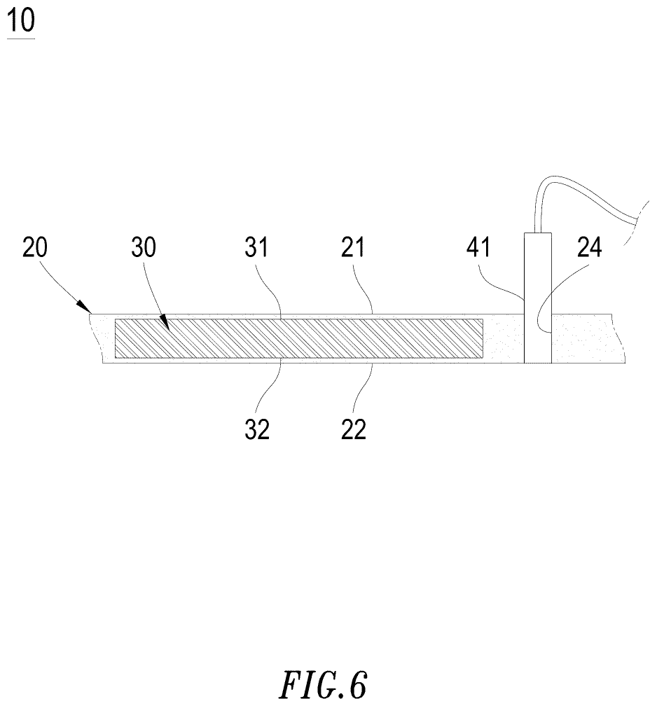

Please refer to FIGS. 5 and 6. FIG. 5 is a three-dimensional schematic diagram of the third embodiment of the present invention. FIG. 6 is a cross-sectional schematic diagram of the third embodiment of the present invention. As shown in the figure, the encapsulated negative magnetic device 10 includes an encapsulation body 20, a plurality of first magnetic elements 30 and a vibration device 40.

The present embodiment differs from the first embodiment in that, the embodiment further includes a vibration device 40, and the encapsulation body 20 includes at least an inserting hole 24. The remaining structures are the same as those of the first embodiment, and thus will not be described in detail. The inserting hole 24 passes through the encapsulation body 20. In other words, the inserting hole 24 has at least two openings on the encapsulation body 20. Regarding the vibration device 40, it includes a vibration motor 41 and a power supply 42. The vibration motor 41 is, for example, but not limited to, any one of an electric motor, a pneumatic motor or a hydraulic motor. The vibration motor 41 is electrically connected to the power supply 42. The power supply 42 is any one of an external power supply and a built-in power supply. The built-in power supply is electrically connected to a circuit board. The circuit board is used to control the power supply mechanism of the built-in power supply, so as to adjust the vibration time or vibration intensity of the vibration motor 41 by adjusting the power supply time or the power supply current. Therefore, when in use, the vibration motor 41 can be inserted into the inserting hole 24, and the S-pole corresponding surface 21 is attached to the human skin, especially the affected part. The negative magnetic effect provides the affected part with the effects of restoring, alkalizing, relaxing, calming, and resisting pressure. When the vibration motor 41 is operated, each first magnetic element 30 can be vibrated to expand the magnetic field function. By striking each first magnetic element 30, the magnetic field can be strengthened to penetrate into the skin and muscles.

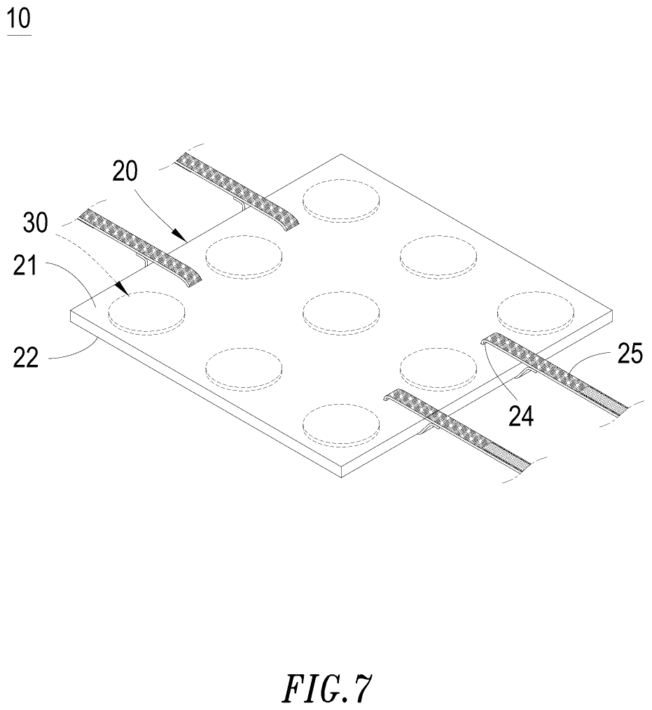

Please refer to FIG. 7. FIG. 7 is a three-dimensional schematic diagram of another configuration of the third embodiment of the present invention. It is worth mentioning that the inserting hole 24 can also be provided with a bandage 25, and the encapsulation body 20 is fixed to the human body through bandage 25. More specifically, one end of the bandage 25 can pass through the inserting hole 24, and then the bandage 25 is tied or wrapped around the affected part of the human body, and the bandage 25 is fixed by tying, gluing, buckling, etc. In order to fix the bandage 25, a fixing structure is provided on the bandage 25. The fixing structure is, for example, but not limited to, a buckle or Velcro.

### Fourth Embodiment

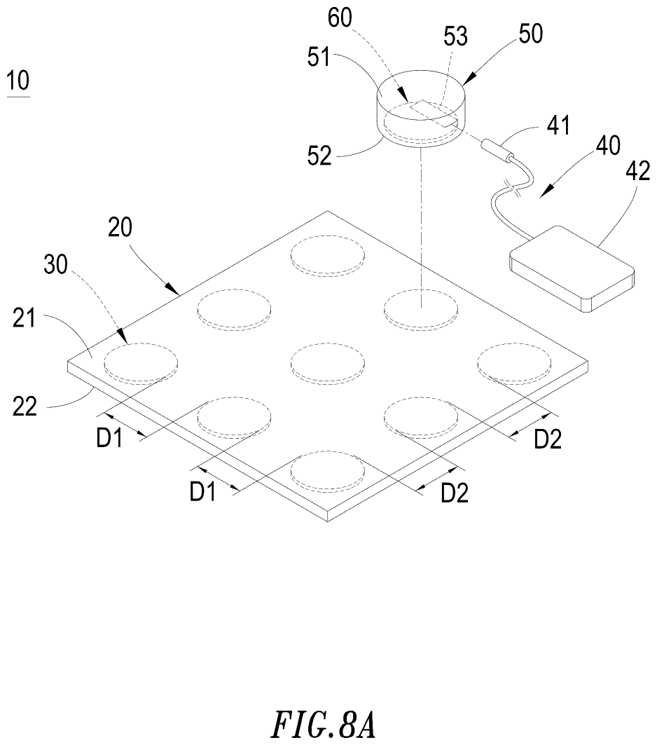

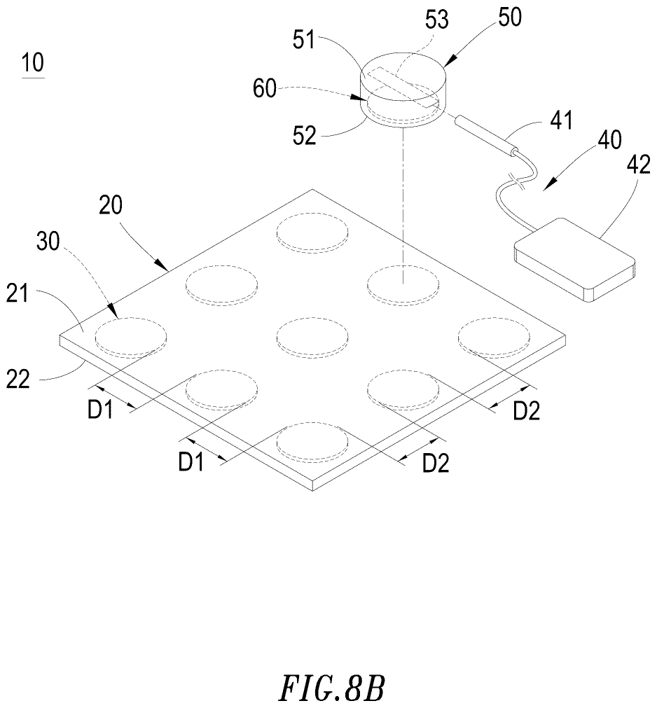

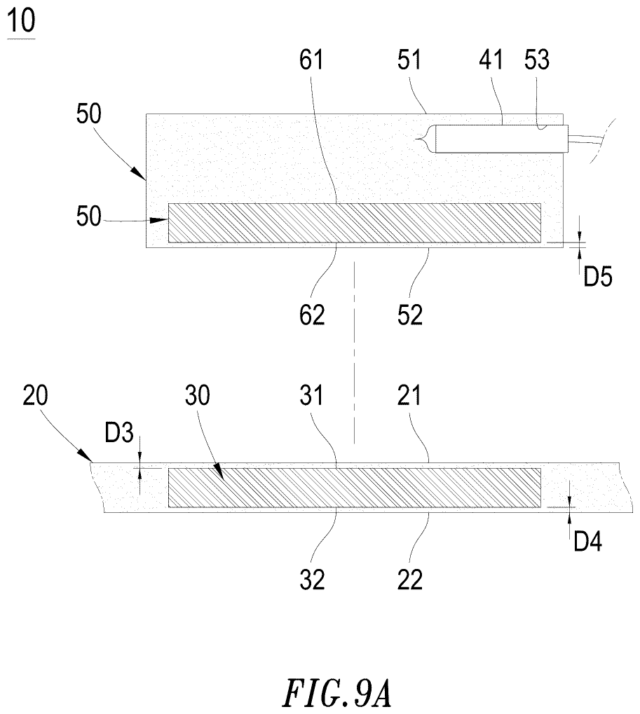

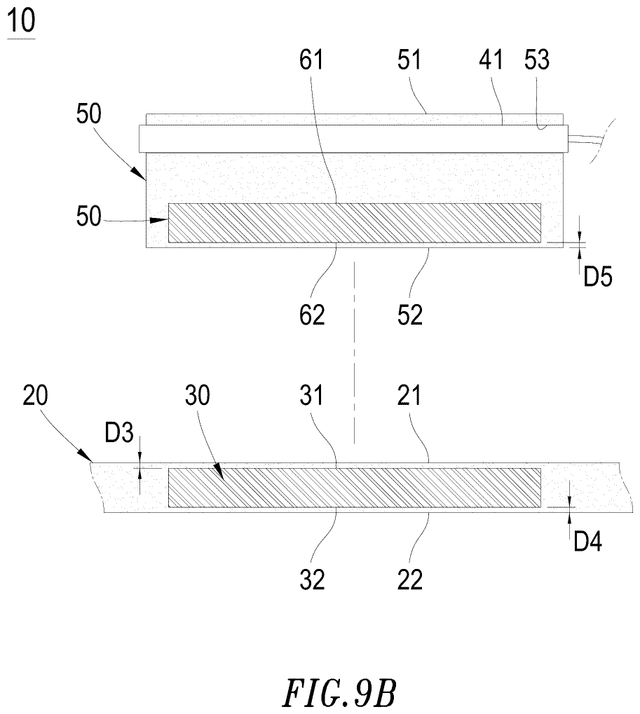

Please refer to FIGS. 8A and 9A. FIG. 8A is a three-dimensional schematic diagram of the fourth embodiment of the present invention. FIG. 9A is a cross-sectional schematic diagram of the fourth embodiment of the present invention. As shown in the figure, the encapsulated negative magnetic device 10 includes an encapsulation body 20, a plurality of first magnetic elements 30, a vibration device 40, an additional encapsulation body 50 and at least one second magnetic element 60.

The present embodiment differs from the first embodiment in that, the present embodiment further includes a vibration device 40, an additional encapsulation body 50 and a second magnetic element 60. The encapsulation body 20 and the plurality of first magnetic elements 30 of the embodiment are the same as those of the first embodiment, and thus will not be described in detail.

Regarding the vibration device 40, it includes a vibration motor 41 and a power supply 42. The vibration motor 41 is, for example, but not limited to, any one of an electric motor, a pneumatic motor or a hydraulic motor. The vibration motor 41 is electrically connected to the power supply 42. The power supply 42 is any one of an external power supply and a built-in power supply. The built-in power supply is electrically connected to a circuit board. The circuit board is used to control the power supply mechanism of the built-in power supply, so as to adjust the vibration time or vibration intensity of the vibration motor 41 by adjusting the power supply time or the power supply current.

The additional encapsulation body 50 is made of silicone, and it has an S-pole corresponding surface 51, an N-pole corresponding surface 52 and a fixed slot 53. The S-pole corresponding surface 51 and the N-pole corresponding surface 52 are located on two opposite side surfaces of the additional encapsulation body 50. The fixed slot 53 does not penetrate the additional encapsulation body 50. In other words, the fixed slot 53 has only one opening on the additional encapsulation body 50. In other embodiments, the fixed slot 53 may also penetrate the additional encapsulation body 50. In other words, the fixed slot 53 only has at least two openings on the additional encapsulation body 50 (as shown in FIGS. 8B and 9B). A second magnetic element 60 is disposed inside the additional encapsulation body 50. The second magnetic element 60 is a permanent magnet. The second magnetic element 60 has a second S-pole surface 61 and a second N-pole surface 62.

In one embodiment, each second S-pole surface 61 is located on the same side of the S-pole corresponding surface 51. That is, each second N-pole surface 62 is located on the same side of the N-pole corresponding surface 52. In order to prevent each second magnetic element 60 from losing its magnetic attraction effect due to the isolation of the additional encapsulation body 50, a N-pole effective magnetic spacing D5 is provided between each second N-pole surface 62 and the N-pole corresponding surface 52, and the effective magnetic spacing D5 is not greater than 10 mm. In this way, the additional encapsulation body 50 can cover each second magnetic element 60 and maintain the magnetic attraction effect of each second magnetic element 60.

As such, when in use, the vibration motor 41 can be inserted into the fixed slot 53, and the S-pole corresponding surface 51 of the additional encapsulation body 50 is attached to the N-pole corresponding surface 22 of the encapsulation body 20. Accordingly, the second magnetic element 60 can be adsorbed on one of the first magnetic element 30, and then the S-pole corresponding surface 21 of the encapsulation body 20 is attached to the human skin, especially the affected part. The negative magnetic effect provides the affected part with the effects of restoring, alkalizing, relaxing, calming, and resisting pressure. When the vibration motor 41 is operated, the second magnetic element 60 and each first magnetic element 30 can be vibrated to expand the magnetic field function. By striking each first magnetic element 30, the magnetic field can be strengthened to penetrate into the skin and muscles.

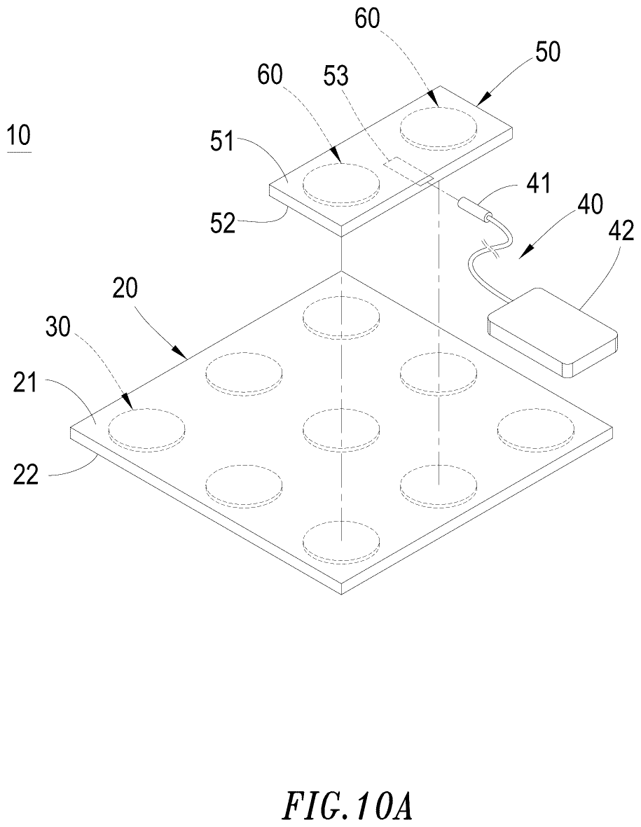

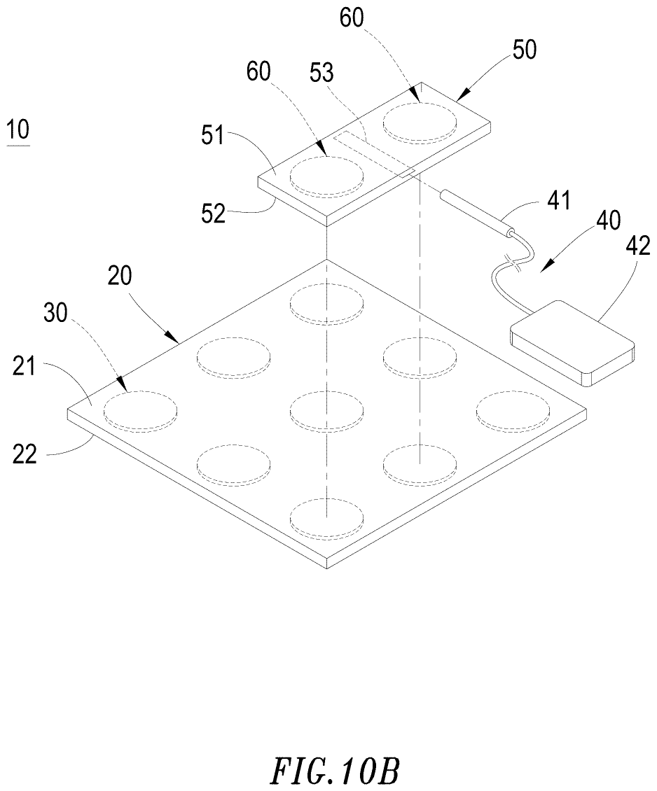

Please refer to FIG. 10A. FIG. 10A is a three-dimensional schematic diagram of another configuration of the fourth embodiment of the present invention. It is worth mentioning that two second magnetic elements 60 can be provided inside the additional encapsulation body 50. When the S-pole corresponding surface 51 of the additional encapsulation body 50 is attached to the N-pole corresponding surface 22 of the encapsulation body 20, each second magnetic element 60 can be adsorbed on two of the first magnetic elements 30. In other embodiments, there can be more than two second magnetic elements 60. As long as the additional encapsulation body 50 is attached to the encapsulation body 20 by adsorbing the first magnetic element 30 through the second magnetic element 60, it falls within the scope of this embodiment. It is worth mentioning that, in this embodiment, the fixed slot 53 does not penetrate the additional encapsulation body 50. In other words, the fixed slot 53 has only one opening on the additional encapsulation body 50. In other embodiments, the fixed slot 53 may also penetrate the additional encapsulation body 50. In other words, the fixed slot 53 only has at least two openings on the additional encapsulation body 50 (as shown in FIG. 10B).

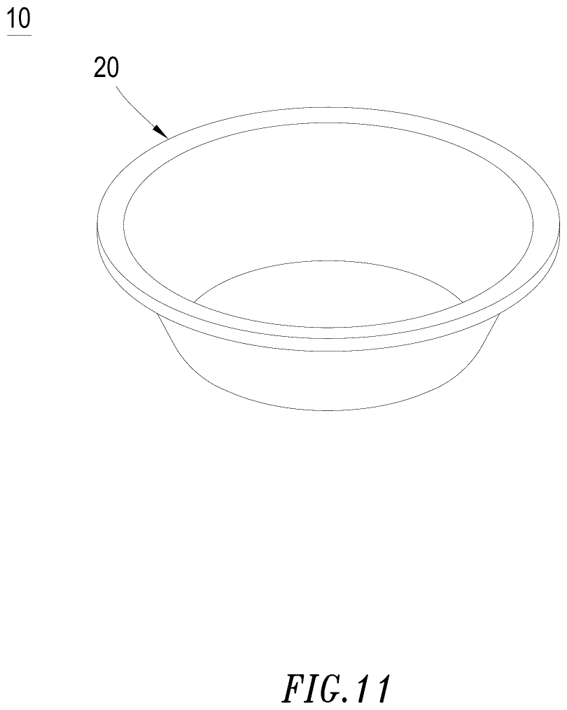

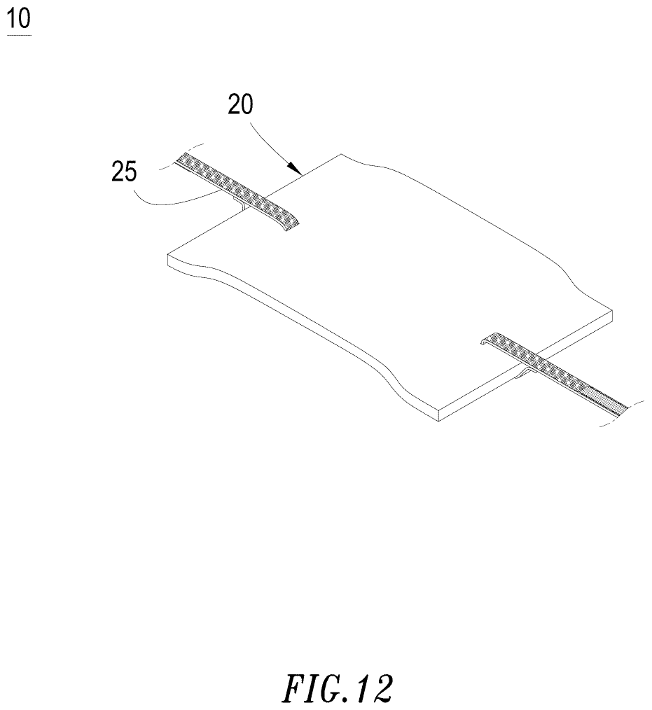

Since the encapsulation body 20 can be formed into various shapes and structures through a mold, the encapsulated negative magnetic device 10 of the present invention can be made into various protective support or pad body, such as a seat cushion (as shown in FIG. 1), a head guard (as shown in FIG. 11), a wrist guard (as shown in FIG. 12), an eye guard/eye mask, a knee guard, an elbow guard . . . etc.

Compared with other conventional technologies, the encapsulated negative magnetic device 10 provided by the present invention can provide the following advantages:
1. By encapsulating the first magnetic element 30 in the encapsulation body 20, the encapsulation body 20 is made of silicone material, which is a flexible material. Therefore, when the encapsulation body 20 is attached to human skin, it can easily fit closely with the skin surface at various locations, and is suitable for various parts of the human body. In addition, the encapsulation body 20 can also be made into various shapes and structures, and can be made into various protective support or pad bodies according to ergonomics. Therefore, the products are diverse.
2. The present invention can also cooperate with the vibration device 40 to strengthen the negative magnetic field of the first magnetic element 30 through vibration and penetrate deep into the skin and muscles. Since the vibration motor 41 can be completely covered by the encapsulation body 20 or the additional encapsulation body 50, it can effectively offset the sound generated during vibration, thereby achieving a noise reduction effect.
3. Because the present invention does not generate an electromagnetic field by driving magnets through power supply, it does not generate electromagnetic waves, and thus will not have any adverse effects on the human body.

The above-mentioned embodiment are only preferred embodiment of the present invention and are not intended to limit the present invention. In the implementation of the present invention, some of the details disclosed in the embodiment are necessary for the purpose of clarifying the disclosure of the specification. Persons skilled in the art should understand that these details are non-essential and should not limit the present invention. Any person who is familiar with the general knowledge in this technical field and understands the aforesaid technical features and embodiments of the present invention, and makes equal changes or embellishments within the spirit and scope of the present invention, shall still fall within the scope of the present invention. The scope of patent protection for the invention shall be as defined in the claim attached hereto.

What is claimed is:

1. An encapsulated negative magnetic device, comprising:
an encapsulation body, made of a flexible material, having an S-pole corresponding surface and an N-pole corresponding surface; and
a plurality of first magnetic elements, encapsulated in the encapsulation body, wherein the first magnetic elements all have a first S-pole surface and a first N-pole surface, the first S-pole surfaces are all located on the same side of the S-pole corresponding surface, and the first N-pole surfaces are all located on the same side of the N-pole corresponding surface.

2. The encapsulated negative magnetic device according to claim 1, wherein a flexible material of the encapsulation body is silicone, and the first magnetic elements are permanent magnets.

3. The encapsulated negative magnetic device according to claim 1, wherein a longitudinal spacing is provided between every two longitudinally adjacent magnetic elements, and a transverse spacing is provided between every two transversely adjacent first magnetic elements.

4. The encapsulated negative magnetic device according to claim 1, wherein a S-pole effective magnetic spacing is provided between the first S-pole surfaces and the S-pole corresponding surface, the S-pole effective magnetic spacing is not greater than 10 mm, a N-pole effective magnetic spacing is provided between the first N-pole surfaces and the N-pole corresponding surface, and the N-pole effective magnetic spacing is not greater than 10 mm.

5. The encapsulated negative magnetic device according to claim 1, wherein the encapsulation body comprises at least one slot, the slots do not penetrate the encapsulation body, the slots are used to fix a vibration device, the vibration device comprises a vibration motor and a power supply, the vibration motor is electrically connected to the power supply, and the vibration motor can be inserted into a slot.

6. The encapsulated negative magnetic device according to claim 1, wherein the encapsulation body comprises at least one inserting hole, the inserting holes all penetrate the encapsulation body, the inserting holes are used to fix a vibration device or a bandage, the vibration device comprises a vibration motor and a power source, the vibration motor is electrically connected to the power supply, and the vibration motor can be inserted into an inserting hole.

7. The encapsulated negative magnetic device according to claim 1, further comprising an additional encapsulation body, wherein a material of the additional encapsulation body is a flexible material, the additional encapsulation body comprises a S-pole corresponding surface, an N-pole corresponding surface and at least one fixed slot, the additional encapsulation body encapsulates at least one second magnetic element, the second magnetic elements comprise a second S-pole surface and a second N-pole surface, the second S-pole surfaces are all located on the same side of the S-pole corresponding surface of the additional encapsulation body, the second N-pole surfaces are all located on the same side of the N-pole corresponding surface of the additional encapsulation body, the fixed slots are used to fix a vibration device, the vibration device comprises a vibration motor and a power supply, the vibration motor is electrically connected to the power supply, and the vibration motor can be inserted into a fixed slot.

8. The encapsulated negative magnetic device according to claim 7, wherein an N-pole effective magnetic spacing is provided between the second N-pole surfaces and the N-pole corresponding surface of the additional encapsulation body, and the N-pole effective magnetic spacing is not greater than 10 mm.

9. The encapsulated negative magnetic device according to claim 7, wherein a flexible material of the additional encapsulation body is silicone, and the second magnetic elements are permanent magnets.
